(19) United States
(12) Patent Application Publication
(10) Pub. No.: US 2010/0081984 A1
(43) Pub. Date: Apr. 1, 2010

(54) Garment with Silicone Transpiring Band and Method for Making the Same

(75) Inventors: Marco Coffinardi, Bagnolo Mella (Brescia) (IT); Giulia Coffinardi, Bagnolo Mella (Brescia) (IT); Luciano Coffinardi, Bagnolo Mella (Brescia) (IT); Monica Coffinardi, Bagnolo Mella (Brescia) (IT)

Correspondence Address: SHOEMAKER AND MATTARE, LTD, 10 POST OFFICE ROAD - SUITE 100, SILVER SPRING, MD, 20910, US

(73) Assignee: GRUPPO DECORTEX S.R.L., Bagnolo Mella (Brescia) (IT)

(21) Appl. No.: 12/528,030

(22) PCT Filed: Feb. 27, 2007
(86) PCT No.: PCT/IT07/00138

§ 371 Date: Oct. 9, 2009

(51) Int. Cl. A61F 13/00; A43B 17/00; A41D 19/00; B05D 5/00

(52) U.S. Cl. 602/63; 2/239; 2/167; 427/288

(57) ABSTRACT

The invention relates to a garment, such as a sock, a short sock, an ankle sock having a fabric portion suitable to be worn on a body part, such as a limb, and at least one silicone band on an area of said fabric portion, such as having an anti-slip function, wherein a plurality of transpiring through openings is formed in said silicone band.

### FIELD OF THE INVENTION

The object of the present invention is a garment, such as a sock, a short sock, stockings, an ankle sock or the like having at least one silicone band particularly though not exclusively provided in an inner part thereof to prevent the garment from slipping down a limb wearing the latter.

### PRIOR ART

In patent application No. WO2006011168 in the name of the same Applicant a garment has been described, which has silicone inserts and a method for making the same. The method provides laying the garment on a support, positioning a thin plate on the flat garment which carries openings corresponding to the shape of the desired inserts to be applied to the garment fabric, and coating a silicone rubber-based mixture having a suitable viscosity on the plate by means of a specific spatula. The silicone mixture passes through the openings of the plate and adheres to the garment. After the silicone mixture has been coated, the thus-obtained garment is heated for the silicone rubber to be cured, and then hardened. Practically, the silicone inserts are applied to the garment by means of a technique similar to screen-printing. The operation of coating the silicone mixture can be thus defined as a printing operation of the silicone inserts.

In an embodiment described in the above-cited document, the silicone insert consists of one or more parallel bands that are applied on the inner edge of a sock, stockings, ankle sock or the like, in order to prevent them from slipping down the leg. In other words, the silicon band is a friction means directly acting on the skin.

In order to obtain the desired and long-lasting hold effect, the silicone band is a continuous band. It is not sufficient, in fact, as it had been previously proposed, to apply individual silicone inserts to the inner edge of the garment, such as in the form of points obtained by depositing a silicone drop by means of a sort of gun.

The silicone continuous band, though fully achieving the desired effect, however, suffers from the drawback that, due to the non-transpiring structure of the silicone mixture, the underlying skin begins to sweat after a certain period of wearing the garment.

It is thus the object of the present invention to overcome this drawback, without however affect the hold of the silicone band.

### SUMMARY OF THE INVENTION

This object is achieved by means of a garment according to claim 1 and by means of a method for making the same according to claim 15.

### BRIEF DESCRIPTION OF THE DRAWINGS

The characteristics and advantages of the present invention will be however understood from the description given below of a preferred exemplary embodiment thereof, which is given as non-limiting indication, with reference to the attached figures, in which:

FIG. 1 shows a women sock with transpiring silicone bands;

FIG. 2 shows an enlarged detail of the inner part of the sock, at the level of the silicone bands;

FIG. 3 shows a plate for printing the silicone bands;

FIG. 4 shows a shaped support on which the garment is fitted;

FIG. 5 shows a top perspective view of a part of the print station of the silicone bands;

FIG. 6 shows a bottom perspective view of the print station;

FIG. 7 shows a detail of a silicone band on stockings according to a different embodiment; and

FIG. 8 shows a detail of the plate for making the silicone bands of the preceding figure.

### DETAILED DESCRIPTION OF THE INVENTION

The present invention is generally directed to any garment, such as a sock, a short sock, an ankle sock, a bandage, a glove, having at least one fabric portion 11 suitable to be worn on a body part, such as a limb, and at least one silicone band 12 applied to an area of said fabric.

Herein below, by silicone band applied to the fabric is meant both a substantially continuous individual area made of silicone material having such an extension as to allow obtaining a plurality of transpiring openings to be formed therein in accordance with the invention, and a plurality of individual silicone areas arranged such as to define a silicone band or stripe.

By silicone is meant a mixture comprising at least one silicone rubber. Preferably, said mixture comprises at least another component. Advantageously, this further component is a catalyst acting as a hardening agent. As will be better described below, in fact, the starting silicone mixture has such a viscosity (such as 70,000 to 90,000 mPa.s) as to be applicable to the garment fabric by means of a coating action. The catalyst subsequently causes the solidification of the silicone mixture. This solidification process can be considerably accelerated by subjecting the just-printed garment to a heating process.

The present invention is particularly directed to a garment, such as a sock, a short sock, an ankle sock, having a tubular body 11 made of fabric, suitable to be worn on a limb and at least one silicone band 12 on an inner portion of said tubular body that is suitable to prevent the garment from slipping down the limb.

With reference to said figures, with 10 has been designated a women's sock having at least one silicone band 12 within the opened end portion 16 thereof. This silicone band 12 has the function of preventing the sock from slipping down the limb when the sock is being worn, which function is mainly carried out due to the friction between the band silicone material and the skin.

According to the invention, a plurality of transpiring through openings 14 is provided in the silicone band 12. By transpiring through opening is meant that in the silicone band is formed a silicone-free area having a negligible section relative to the band size. In other words, any transpiring opening is surrounded, possibly except for a transpiring channel which will be better described below, by the silicone material.

In still other words, considering the surface occupied by the band, the ratio of the silicone-free surface to that occupied by silicone is negligible.

In fact, it is crucial to make the silicone material layer as continuous as possible, in order to obtain a high slipping resistance.

According to a very advantageous embodiment, the transpiring openings are in the form of holes. In other words, they are substantially point-like openings.

In accordance with an embodiment, the silicone band 12 has a thickness ranging between 0.2 mm and 0.8 mm, preferably 0.5 mm.

Preferably, the transpiring holes 14 have a diameter ranging between 0.4 mm and 1 mm, for example 0.6 mm.

Advantageously, the silicone band 12 is a continuous band at least along a substantially semi-annular portion of the inner annular portion of the tubular body 11. In other words, considering the lower size of the band to be crosswise, i.e. the one parallel to the axis of the tubular body 11, despite the provision of transpiring holes 14, the band 12 is free of crosswise discontinuities. In still other terms, a silicon-free pathway does not exist, which crosses the band placing the fabric portions of the tubular body separated by the band in communication with each other.

Advantageously, the silicone band 12 is a continuous band all over the inner annular portion, except for two interruptions 18 extending along two diametrally opposite directrixes of the tubular body 11. As will be better explained below, these interruptions are due to the method for applying silicone to the garment, which provides laying the previously-formed tubular body on a flat surface and sequentially coating the silicone on both faces of the flattened tubular body. It is understood that the longitudinal edges of the flattened tubular body cannot be coated with silicone.

In accordance with an embodiment, in the silicone band 12 is formed at least one array of transpiring holes 14 developing in the length direction of the band, i.e. parallel to the long sides thereof. The adjacent holes 14 of said array of holes are connected to each other by a transpiring channel 15 extending between the ends of the band, i.e. between the short sides.

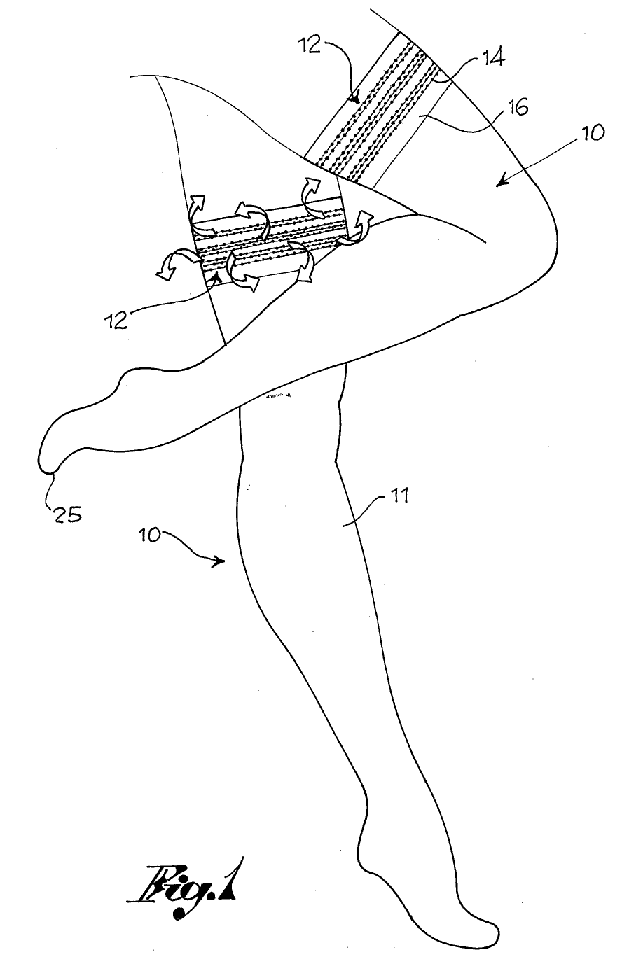

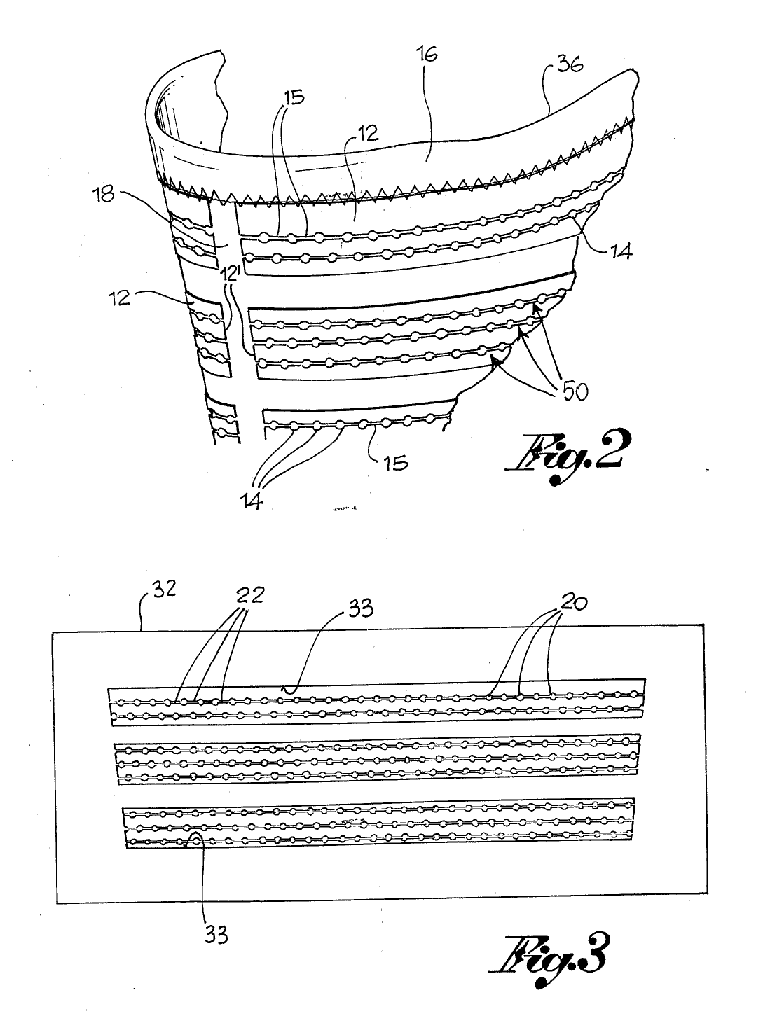

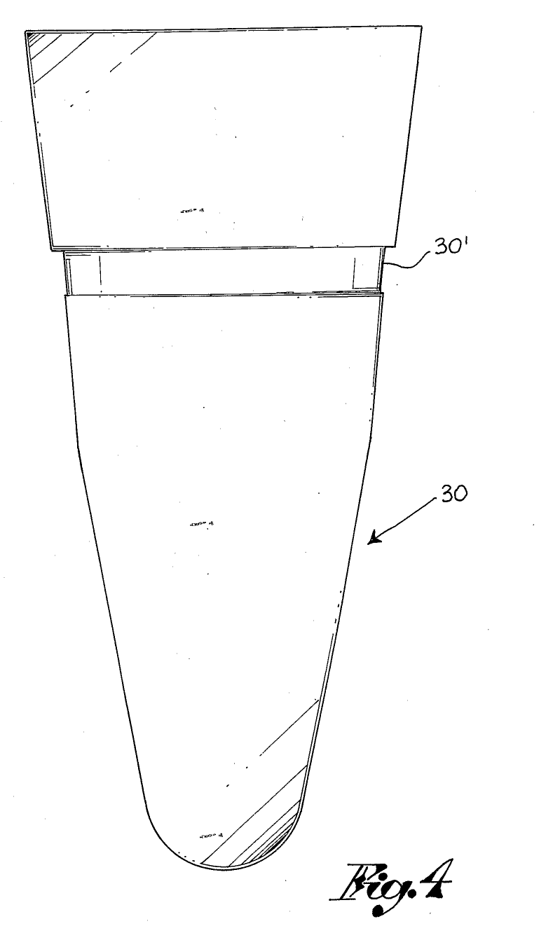

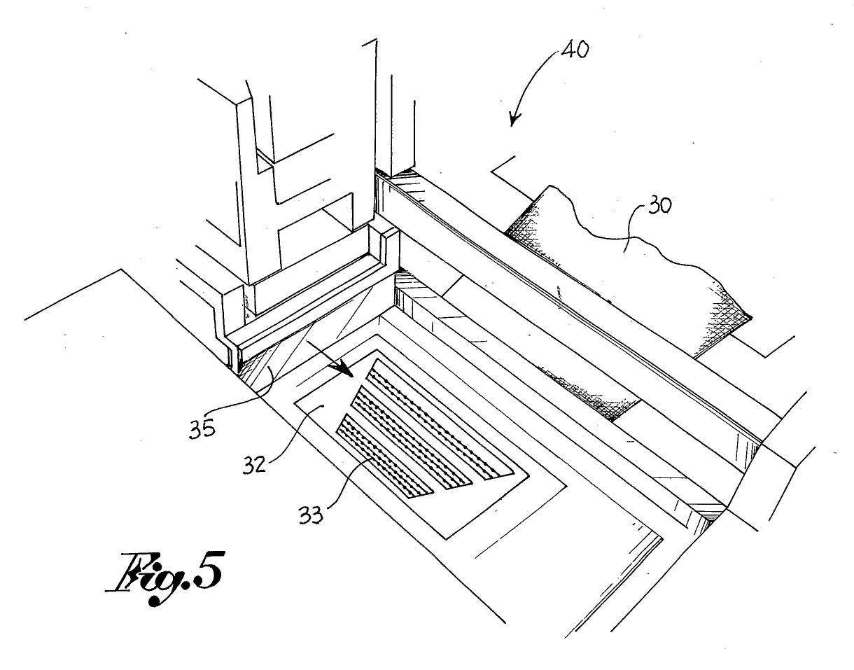

In the example shown in FIG. 1-3, each silicone band 12 carries parallel rows 50 of transpiring holes 14. The holes in each row 50 are connected to each other by a longitudinal transpiring channel 15 opened at the ends of the band 12.

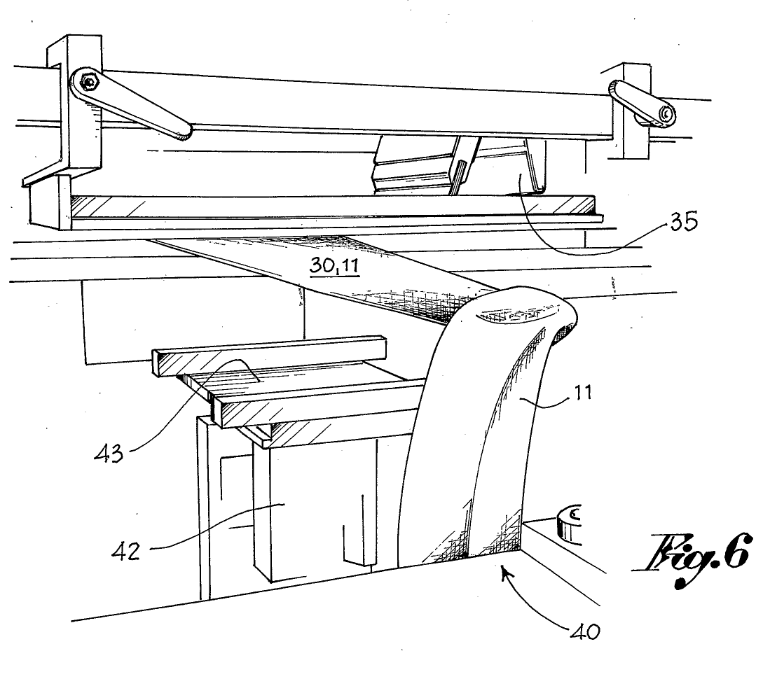

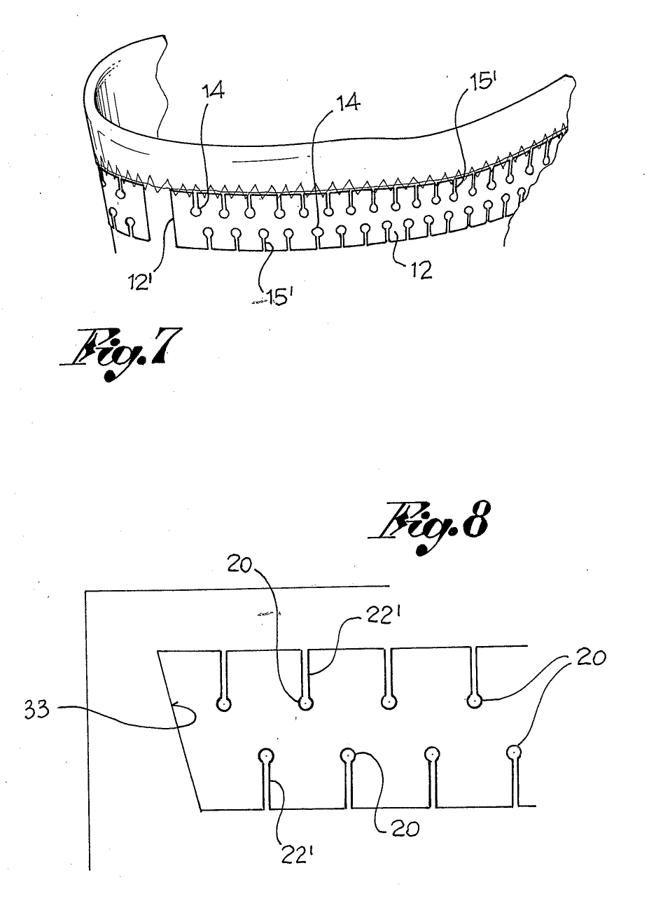

In accordance with a variant embodiment illustrated in FIGS. 6 and 7, each transpiring hole 14 is connected to the closest fabric area thereto, via at least one transpiring channel 15′. In other words, to each hole 14 is associated at least one channel 15′ thereof, which is opened on one of the two long sides of the band 12. These channels 15′ are then substantially oriented in the orthogonal direction to the long sides of the band, i.e. transversal thereto.

Obviously, combinations of the two embodiments of the transpiring channels 15, 15′ are possible. In any case, it should be observed that the provision of these channels 15, 15′ essentially derives, as will be explained herein below, from the method for making the transpiring holes 14. In brief, as the transpiring holes are obtained by means of the provision of corresponding cores 20 that prevent the silicone mixture from depositing on the underlying fabric, the transpiring channels 15, 15′ are silicone-free areas corresponding to thread-like elements 22 supporting said solid portions 20.

Preferably, each transpiring channel 15, 15′ has a width equal to or lower than 0.5 mm, more preferably equal to or lower than 0.3 mm. The selection of this width depends on the desired effect to be obtained. For example, when the transpiring effect of the silicone band 12 is preferred, a width of the thread-like elements 22 can be selected, such as to make the transpiring channels also suitable for significantly contributing to skin transpiration. On the other hand, when the provision of the transpiring holes 14 is already sufficient per se in order to ensure the desired transpiring effect and the anti-slip hold effect is preferred for the garment, the lowest possible thickness will be selected for the thread-like elements 22, such that, after said thread-like elements have been removed, the just-coated silicone mixture defining the transpiring channels 15, 15′ will tend to recompose thus occluding these channels.

According to a further aspect of the invention, the top portion 16 carrying at least one band 12 has a substantially conical shape, such as to be adapted to the anatomy of the limb on which it is intended to be worn. In accordance with a preferred embodiment, the tubular body 11 has a top portion 16 with at least two silicone bands 12.

Advantageously, the at least two bands 12 have a decreasing circumference (or length, when seen as individual silicone areas) starting from the top of the tubular body 11. Advantageously, furthermore, the bands have a trapezoid shape with the short sides 12′ inclined parallel to the conical shape of the top portion 16 of the tubular body 11.

Such a configuration of the tubular body and silicone bands 12 thereof allows obtaining a same and high anti-slip hold all over the top portion 16, without applying an excessive pressure on a limited area of the limb.

Advantageously, the tubular body 11 is formed as one piece. For example, the tubular body 11 is closed at one end 25 and has a stitching only at said closed end.

In accordance with a general embodiment, the above-described method for making the garment comprises the steps of:

laying a fabric portion 11 of the garment on a flat support surface 30,

placing a plate 32 on said fabric portion, in which plate at least one opening 33 is provided having a shape equal to that of the silicone band 12 intended to be applied to the tissue,

coating the silicone-based mixture on said plate 32 such that said mixture deposits on the fabric at said opening 33;

consolidating the mixture deposited on the fabric by means of a drying process.

As stated above, the transpiring openings 14 are obtained by providing corresponding cores 20 within the opening 33 in the plate 32, which cores are connected to the edges of the opening 33, either directly or via adjacent cores, by support cores 22 providing the transpiring channels 15, 15′.

In the example in FIG. 1-3, said cores 20 are point-like cores 20 arranged in rows within the opening such that the support cores 22 extend substantially parallel to the long sides of the opening.

In the variant embodiment in FIGS. 7 and 8, the cores 20 are point-like cores 20 arranged within the opening 33 such that the support cores 22′ extend substantially orthogonal to the long sides of the opening.

Advantageously, the cores 20 of the transpiring openings 14 and the support cores 22, 22′ are formed integral with the plate 32. For example, the opening 33 carrying the cores 2, 22, 22′ is obtained as a shaped opening from a solid plate by means of a photogravure process.

Advantageously, the silicone mixture is coated on the plate 32 with a scrapers or spatulas system 35. Preferably, the direction in which this scrapers system moves is parallel to the direction in which the support cores 22, 22′ extend, such as to avoid breaking these cores, which are thin.

In accordance with an embodiment of the method for making the garment, the tubular body 11 is fitted on a flattened shaped support 30 capable of rotating about an axis thereof parallel to that of the tubular body 11, such as to sequentially receive the plate 32 on both sides thereof.

When the garment 10 has a lap 36 along the edge of the top portion 16 of the tubular body 11, the shaped support 30 advantageously has a lowered area 30′ suitable to accommodate said edge with flap 36 such as to compensate the thickness difference between said edge and the remainder of the tubular body. In doing so, the rest surface of the plate 32 is however perfectly flat.

The method for providing the garment is implemented with an automated plant, as described for example in the prior patent application in the name of the same applicant.

This plant comprises at least one station for inserting the garment on the shaped support 30, at least one station for removing the garment from said support, at least one print station 40, in which the silicone mixture is applied to the fabric, at least one drying station, and at least one cooling station.

The print station 40 comprises means for carrying out the rotation of the support 30 about the axis thereof such as to carry out the print of the silicone mixture on both sides of the garment being fitted on the shaped support 30.

During the step of coating the silicone mixture, the shaped support 30 is rested on an abutment 42 acting on the opposite side relative to the rest side of the plate 32. Advantageously, said abutment 42 has a recess or notch 43 at the portion of fabric carrying the silicone bands 12, such as to avoid contacting said silicone bands that have just deposited on the fabric. Thereby, it is not required to wait for the hardening of the bands 12 before carrying out the print on the other side of the garment, thus considerably reducing the time required for making the garment.

In accordance with an embodiment, the plate 32 consists of a sheet metal, such as brass. Preferably, this laminate has a thickness of about 0.5-1 mm.

It should be noted that the shaped support 30 is configured such that, when the garment is fitted on said support, it closely adheres thereto and is subjected, due to the elasticity of the fabric, a considerable increase in the surface extension thereof. This surface extension increase is substantially equal to that occurring when the garment is worn on the limb. Accordingly, as the coating is carried out in these dimensional conditions of the fabric, it is ensured that when the garment is worn, the silicone band 12 will be however continuous and smooth, and will thus carry out its anti-slip hold function at the best.

Tests carried out on a sock have shown, as compared with a stock with a traditional band, about 50% extra hold.

It should be noted, in this regard, that the provision of the transpiring holes 14 allows applying wider (in the crosswise direction) silicone bands 12 to the garment, and thus capable of providing a higher hold, as compared with a band not provided with transpiring effect. In fact, the width of the silicone bands without transpiring holes is restrained by the skin intense sweating caused by this band.

Finally, it should be noted that the provision of the cores 20, 22, 22′ allows, when the plate 32 is pressed on the fabric, avoiding that the fabric at the opening 33 is raised. Thereby, at the silicone bands, the garment does not form excessively raised and discontinuous areas relative to the remainder of the fabric. The aesthetic appearance and the touch sensation are, accordingly, not significantly altered by the provision of the silicone bands.

1-23. (canceled)

24. A garment, such as a sock, a short sock, an ankle sock, a bandage, a glove, having a tubular body made of fabric, which is suitable to be worn on a limb and having a top portion with a substantially conical shape, such as to be adapted to the anatomy of the limb on which it is intended to be worn, wherein at least one silicone band is applied to an inner portion of said tubular body such as to prevent the garment from slipping down said limb, and wherein a plurality of transpiring through openings is formed in said silicone band, characterised in that said top portion carries at least two silicone bands having a decreasing length starting from the top of the garment.

25. The garment according to claim 24, wherein said transpiring openings are in form of holes.

26. The garment according to claim 24, wherein said transpiring holes have a diameter ranging between 0.4 mm and 1 mm, preferably 0.6 mm.

27. The garment according to claim 24, wherein said silicone band has a thickness ranging between 0.2 mm and 0.8 mm, preferably 0.5 mm.

28. The garment according to claim 24, wherein said silicone band is a continuous band at least along a substantially semi-annular portion of said inner portion of the tubular body.

29. The garment according to claim 28, wherein said silicone band is a continuous band along an inner annular portion of the tubular body, except for two interruptions extending along two diametrally opposite directrixes of the tubular body.

30. The garment according to claim 24, wherein at least one array of transpiring holes is formed in the silicone band, which develops in the length direction of said silicone band, and wherein the adjacent holes in this array of holes are connected to each other by a transpiring channel extending between the ends of the band.

31. The garment according to claim 24, wherein each transpiring hole is connected to the fabric area closest thereto via at least one transpiring channel.

32. The garment according to claim 30, wherein said transpiring channel has a width equal to or lower than 0.5 mm, preferably equal to or lower than 0.3 mm.

33. The garment according to claim 24, wherein the tubular body is provided as one piece.

34. The garment according to claim 24, wherein the tubular body is closed at one end and has a stitching only at said closed end.

35. A method for providing a garment according to claim 24, comprising the steps of:
laying at least one fabric portion of the garment on a flat support surface,
placing a plate on said fabric portion, in which plate at least one opening is provided having a shape equal to that of the silicone band intended to be applied to the fabric, wherein transpiring openings are obtained in the silicone band by providing corresponding cores within said opening in the plate, which cores are connected to the edges of said opening, either directly or via adjacent cores, by support cores providing the transpiring channels;
coating a silicone-based mixture on said plate such that said mixture deposits on the fabric at said opening;
consolidating the mixture deposited on the fabric by means of a drying process.

36. The method according to claim 35, wherein the cores of the transpiring openings are arranged within the opening in the plate such that said support cores extend substantially parallel to the long sides of the opening.

37. The method according to claim 35, wherein the cores of the transpiring openings are arranged within the opening in the plate such that the respective support cores extend substantially orthogonal to the long sides of the opening

38. The method according to claim 35, wherein the silicone mixture is coated in a direction parallel to the support cores.

39. The method according to claim 35, wherein the cores are provided integral to the plate.

40. The method according to claim 35, wherein the garment is fitted on a flattened shaped support capable of rotating about an axis thereof such as to receive the plate sequentially on both sides thereof.

41. The method according to claim 40 for making a sock, a short sock, an ankle sock, or similar garment having an edge with a lap, wherein said shaped support has a lowered area suitable to accommodate said edge with flap such as to compensate the thickness thereof and make the rest surface of the plate perfectly flat.

42. The method according to claim 40, wherein during the step of coating the silicone mixture, the shaped support rests on an abutment acting on the opposite side to that of the plate, said abutment having a concave portion at the fabric portion carrying the silicone bands, such as to avoid contacting said silicone bands that have just been deposited on the fabric.
